(19) United States
(12) Patent Application Publication
(10) Pub. No.: US 2025/0228500 A1
(43) Pub. Date: Jul. 17, 2025

(54) ANALYTE SIGNAL PROCESSING DEVICE AND METHODS

(71) Applicant: ABBOTT DIABETES CARE INC., Alameda, CA (US)

(72) Inventor: Martin J. Fennell, Concord, CA (US)

(73) Assignee: ABBOTT DIABETES CARE INC., Alameda, CA (US)

(21) Appl. No.: 19/070,178

(22) Filed: Mar. 4, 2025

Related U.S. Application Data
(63) Continuation of application No. 17/361,089, filed on Jun. 28, 2021, now Pat. No. 12,279,894.
Application No. 17/361,089 is a continuation of application No. 15/953,378, filed on Apr. 13, 2018, now Pat. No. 11,045,147.
Application No. 15/953,378 is a continuation of application No. 15/090,610, filed on Apr. 4, 2016, now Pat. No. 9,968,302.
Application No. 15/090,610 is a continuation of application No. 12/873,294, filed on Aug. 31, 2010, now Pat. No. 9,314,195.
(60) Provisional application No. 61/238,658, filed on Aug. 31, 2009.

(51) Int. Cl. A61B 5/00; A61B 5/145; A61B 5/1473; A61B 5/1486; G01N 15/01; G01N 15/06

(52) U.S. Cl. CPC A61B 5/7228; A61B 5/002; A61B 5/14532; A61B 5/1473; A61B 5/14865; G01N 15/0606; G01N 15/0656; A61B 2560/0209; G01N 15/01

(57) ABSTRACT

Systems configured to perform determining a measurement time period, generating a plurality of clock signals, receiving a plurality of analog signals during the determined measurement time period, the measurement time period establishing a maximum signal resolution associated with the clock and a sigma-delta modulator as a function of analog to digital conversion time to obtain a higher signal resolution, modulating the received plurality of analog signals to generate a frequency data stream over the measurement time period based on the received plurality of analog signals by synchronizing each of the received plurality of analog signals to a corresponding one of the generated plurality of clock signals, and filtering one or more signal artifacts from the frequency data stream over the measurement time period.

### RELATED APPLICATIONS

The present application is a continuation of U.S. patent application Ser. No. 17/361,089 filed Jun. 28, 2021, which is a continuation of U.S. patent application Ser. No. 15/953,378 filed Apr. 13, 2018, now U.S. Pat. No. 11,045,147, which is a continuation of U.S. patent application Ser. No. 15/090,610 filed Apr. 4, 2016, now U.S. Pat. No. 9,968,302, which is a continuation of U.S. patent application Ser. No. 12/873,294 filed Aug. 31, 2010, now U.S. Pat. No. 9,314,195, which claims the benefit of U.S. Provisional Application No. 61/238,658 filed Aug. 31, 2009, entitled “Analyte Signal Processing Device and Methods”, the disclosures of each of which are incorporated herein by reference for all purposes.

### BACKGROUND

The monitoring of the level of glucose or other analytes, such as lactate or oxygen, in certain individuals is vitally important to their health. High or low levels of glucose or other analytes may have detrimental effects. The monitoring of glucose is particularly important to individuals with diabetes, as they must determine when insulin is needed to reduce glucose levels in their bodies or when additional glucose is needed to raise the level of glucose in their bodies.

A conventional technique used by many diabetics for personally monitoring their blood glucose level includes the periodic drawing of blood, the application of that blood to a test strip, and the determination of the blood glucose level using colorimetric, electrochemical, or photometric detection. This technique does not permit continuous or automatic monitoring of glucose levels in the body, but typically must be performed manually on a periodic basis. Unfortunately, the consistency with which the level of glucose is checked varies widely among individuals. Many diabetics find the periodic testing inconvenient and they sometimes forget to test their glucose level or do not have time for a proper test. In addition, some individuals wish to avoid the pain associated with the test. These situations may result in hyperglycemic or hypoglycemic episodes. An in vivo glucose sensor that continuously or automatically monitors the individual's glucose level would enable individuals to more easily monitor their glucose, or other analyte, levels.

A variety of devices have been developed for continuous or automatic monitoring of analytes, such as glucose, in the blood stream or interstitial fluid. A number of these devices use electrochemical sensors which are directly implanted into a blood vessel or in the subcutaneous tissue of a patient. However, these devices are often difficult to reproducibly and inexpensively manufacture in large numbers. In addition, these devices are typically large, bulky, and/or inflexible, and many cannot be used effectively outside of a controlled medical facility, such as a hospital or a doctor's office, unless the patient is restricted in his activities.

The patient's comfort and the range of activities that can be performed while the sensor is implanted are important considerations in designing extended-use sensors for continuous or automatic in vivo monitoring of the level of an analyte, such as glucose. There is a need for a small, comfortable device which can continuously monitor the level of an analyte, such as glucose, while still permitting the patient to engage in normal activities. Continuous and/or automatic monitoring of the analyte can provide a warning to the patient when the level of the analyte is at or near a threshold level. For example, if glucose is the analyte, then the monitoring device might be configured to warn the patient of current or impending hyperglycemia or hypoglycemia. The patient can then take appropriate actions.

### SUMMARY

An analyte signal processing method in one aspect of the present disclosure includes determining a measurement time period, receiving a plurality of signals associated with a monitored analyte level during the determined measurement time period from an analyte sensor, modulating the received plurality of signals to generate a data stream over the measurement time period, and accumulating the generated data stream to determine an analyte signal corresponding to the monitored analyte level associated with the measurement time period.

A signal processing device used for processing analyte related signals in accordance with another aspect includes an analyte sensor interface electronics for receiving a plurality of analyte related signals from an analyte sensor over a measurement time period, a data processing component operatively coupled to the analyte sensor interface electronics for processing the received plurality of analyte related signals, the data processing component including a signal filtering component to filter the received plurality of analyte related signals, a signal conversion component operatively coupled to the signal filtering component to convert the received filtered plurality of analyte related signals to determine an analyte level associated with a monitored analyte level during the measurement time period.

An analyte monitoring system in still another aspect includes an analyte sensor including a working electrode having a sensing layer at least a portion of which is configured for fluid contact with an interstitial fluid under a skin layer, and a data processing unit comprising an application specific integrated circuit (ASIC) in signal communication with the analyte sensor for receiving a plurality of signals related to a monitored analyte level from the sensor over a monitoring time period, the data processing unit including: a signal filtering component to filter the received plurality of signals; and a signal conversion component to convert the received filtered plurality of analyte related signals to determine a corresponding analyte level associated with a monitored analyte level during a measurement time period, said monitoring time period including multiple measurement time periods.

These and other features, objects and advantages of the various embodiments of the present disclosure will become apparent to those persons skilled in the art upon reading the details of the present disclosure as more fully described below.

### BRIEF DESCRIPTION OF THE DRAWINGS

FIG. 1 shows a block diagram of an embodiment of a data monitoring and management system according to the present disclosure;

FIG. 2 shows a block diagram of an embodiment of the transmitter unit of the data monitoring and management system of FIG. 1;

FIGS. 3A and 3B illustrate the sensor/transmitter interface including the analog interface section of the data processing unit 102 of FIG. 2 in one embodiment of the present disclosure;

FIG. 4 shows a block diagram of an embodiment of the receiver/monitor unit of the data monitoring and management system of FIG. 1;

FIG. 5 shows a schematic diagram of an embodiment of an analyte sensor according to the present disclosure; and

FIGS. 6A and 6B show a perspective view and a cross sectional view, respectively, of another embodiment of an analyte sensor.

### INCORPORATED BY REFERENCE

The following patents, applications and/or publications are incorporated herein by reference for all purposes: U.S. Pat. Nos. 5,262,035; 5,262,305; 5,264,104; 5,320,715; 5,543,326; 5,593,852; 6,103,033; 6,120,676; 6,134,461; 6,175,752; 6,284,478; 6,560,471; 6,579,690; 6,591,125; 6,605,200; 6,605,201; 6,650,471; 6,654,625; 6,676,819; 6,746,582; 6,881,551; 6,932,892; 6,932,894; 7,299,082; U.S. Published Patent Application Nos. 2004/0186365, now U.S. Pat. No. 7,811,231; 2005/0182306, now U.S. Pat. No. 8,771,183; 2007/0056858, now U.S. Pat. No. 8,298,389; 2007/0068807, now U.S. Pat. No. 7,846,311; 2007/0227911, now U.S. Pat. No. 7,887,682; 2007/0233013; 2008/0081977, now U.S. Pat. No. 7,618,369; 2008/0161666; and 2009/0054748, now U.S. Pat. No. 7,885,698; U.S. patent application Ser. Nos. 12/131,012; 12/242,823, now U.S. Pat. No. 8,219,173; and Ser. No. 12/363,712, now U.S. Pat. No. 8,346,335; and U.S. Provisional Application Nos. 61/149,639; 61/155,889; 61/155,891; 61/155,893; 61/165,499; 61/230,686; 61/227,967 and 61/238,461.

### DETAILED DESCRIPTION

Before the present disclosure is described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges as also encompassed within the disclosure, subject to any specifically excluded limit in the stated range. Where the stated range includes one or both of the limits, ranges excluding either or both of those included limits are also included in the disclosure.

It must be noted that as used herein and in the appended claims, the singular forms “a”, “an”, and “the” include plural referents unless the context clearly dictates otherwise.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The figures shown herein are not necessarily drawn to scale, with some components and features being exaggerated for clarity.

Generally, embodiments of the present disclosure relate to methods and devices for detecting at least one analyte such as glucose in body fluid. Embodiments relate to the continuous and/or automatic in vivo monitoring of the level of one or more analytes using a continuous analyte monitoring system that includes an analyte sensor at least a portion of which is to be positioned beneath a skin surface of a user for a period of time and/or the discrete monitoring of one or more analytes using an in vitro blood glucose (“BG”) meter and an analyte test strip. Embodiments include combined or combinable devices, systems and methods and/or transferring data between an in vivo continuous system and a BG meter system.

Accordingly, embodiments include analyte monitoring devices and systems that include an analyte sensor—at least a portion of which is positionable beneath the skin of the user—for the in vivo detection, of an analyte, such as glucose, lactate, and the like, in a body fluid. Embodiments include wholly implantable analyte sensors and analyte sensors in which only a portion of the sensor is positioned under the skin and a portion of the sensor resides above the skin, e.g., for contact to a transmitter, receiver, transceiver, processor, etc. The sensor may be, for example, subcutaneously positionable in a patient for the continuous or periodic monitoring of a level of an analyte in a patient's interstitial fluid. For the purposes of this description, continuous monitoring and periodic monitoring will be used interchangeably, unless noted otherwise. The sensor response may be correlated and/or converted to analyte levels in blood or other fluids. In certain embodiments, an analyte sensor may be positioned in contact with interstitial fluid to detect the level of glucose, in which detected glucose may be used to infer the glucose level in the patient's bloodstream. Analyte sensors may be insertable into a vein, artery, or other portion of the body containing fluid. Embodiments of the analyte sensors of the subject disclosure may be configured for monitoring the level of the analyte over a time period which may range from minutes, hours, days, weeks, or longer.

Of interest are analyte sensors, such as glucose sensors, that are capable of in vivo detection of an analyte for about one hour or more, e.g., about a few hours or more, e.g., about a few days of more, e.g., about three or more days, e.g., about five days or more, e.g., about seven days or more, e.g., about several weeks or at least one month. Future analyte levels may be predicted based on information obtained, e.g., the current analyte level at time t₀, the rate of change of the analyte, etc. Predictive alarms may notify the user of a predicted analyte level that may be of concern in advance of the user's analyte level reaching the future level. This provides the user an opportunity to take corrective action.

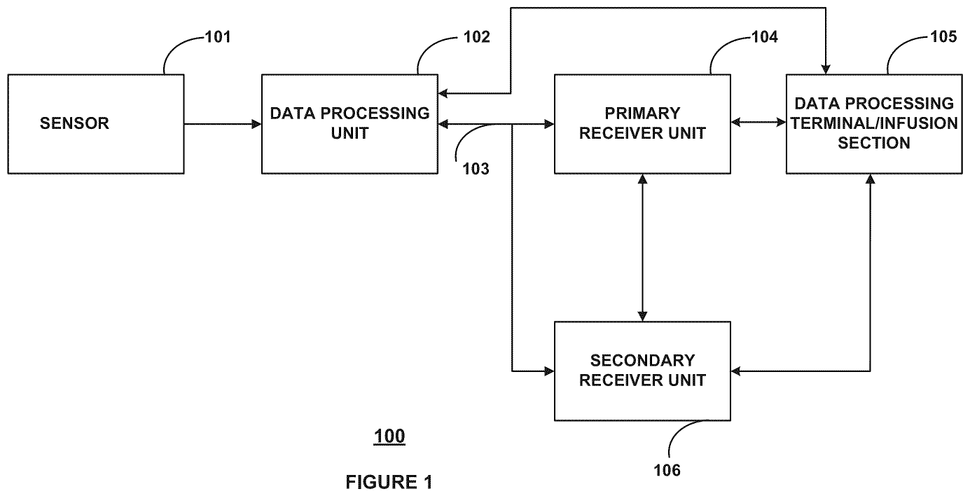

FIG. 1 shows a data monitoring and management system such as, for example, an analyte (e.g., glucose) monitoring system 100 in accordance with certain embodiments. Embodiments of the subject disclosure are further described primarily with respect to glucose monitoring devices and systems, and methods of glucose detection, for convenience only and such description is in no way intended to limit the scope of the disclosure. It is to be understood that the analyte monitoring system may be configured to monitor a variety of analytes at the same time or at different times.

Analytes that may be monitored include, but are not limited to, acetyl choline, amylase, bilirubin, cholesterol, chorionic gonadotropin, creatine kinase (e.g., CK-MB), creatine, creatinine, DNA, fructosamine, glucose, glutamine, growth hormones, hormones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In those embodiments that monitor more than one analyte, the analytes may be monitored at the same or different times.

The analyte monitoring system 100 includes a sensor 101, a data processing unit 102 connectable to sensor 101, and a primary receiver unit 104 which is configured to communicate with data processing unit 102 via a communication link 103. In certain embodiments, primary receiver unit 104 may be further configured to transmit data to a data processing terminal 105 to evaluate or otherwise process or format data received by primary receiver unit 104. Data processing terminal 105 may be configured to receive data directly from data processing unit 102 via a communication link which may optionally be configured for bi-directional communication. Further, data processing unit 102 may include a transmitter or a transceiver to transmit and/or receive data to and/or from primary receiver unit 104 and/or data processing terminal 105 and/or optionally the secondary receiver unit 106.

Also shown in FIG. 1 is an optional secondary receiver unit 106 which is operatively coupled to the communication link and configured to receive data transmitted from data processing unit 102. The secondary receiver unit 106 may be configured to communicate with primary receiver unit 104, as well as data processing terminal 105. The secondary receiver unit 106 may be configured for bi-directional wireless communication with each of primary receiver unit 104 and data processing terminal 105. As discussed in further detail below, in certain embodiments the secondary receiver unit 106 may be a de-featured receiver as compared to the primary receiver, i.e., the secondary receiver may include a limited or minimal number of functions and features as compared with primary receiver unit 104. As such, the secondary receiver unit 106 may include a smaller (in one or more, including all, dimensions), compact housing or embodied in a device such as a wrist watch, arm band, etc., for example. Alternatively, the secondary receiver unit 106 may be configured with the same or substantially similar functions and features as primary receiver unit 104. The secondary receiver unit 106 may include a docking portion to be mated with a docking cradle unit for placement by, e.g., the bedside for night time monitoring, and/or a bi-directional communication device. A docking cradle may recharge a powers supply.

Only one sensor 101, data processing unit 102 and data processing terminal 105 are shown in the embodiment of the analyte monitoring system 100 illustrated in FIG. 1. However, it will be appreciated by one of ordinary skill in the art that the analyte monitoring system 100 may include more than one sensor 101 and/or more than one data processing unit 102, and/or more than one data processing terminal 105. Multiple sensors may be positioned in a patient for analyte monitoring at the same or different times. In certain embodiments, analyte information obtained by a first positioned sensor may be employed as a comparison to analyte information obtained by a second sensor. This may be useful to confirm or validate analyte information obtained from one or both of the sensors. Such redundancy may be useful if analyte information is contemplated in critical therapy-related decisions. In certain embodiments, a first sensor may be used to calibrate a second sensor.

The analyte monitoring system 100 may be a continuous monitoring system, or semi-continuous, or a discrete monitoring system. In a multi-component environment, each component may be configured to be uniquely identified by one or more of the other components in the system so that communication conflict may be readily resolved between the various components within the analyte monitoring system 100. For example, unique IDs, communication channels, and the like, may be used.

In certain embodiments, sensor 101 is physically positioned in or on the body of a user whose analyte level is being monitored. Sensor 101 may be configured to at least periodically sample the analyte level of the user and convert the sampled analyte level into a corresponding signal for transmission by data processing unit 102. Data processing unit 102 is coupleable to sensor 101 so that both devices are positioned in or on the user's body, with at least a portion of the analyte sensor 101 positioned transcutaneously. The data processing unit may include a fixation element such as adhesive or the like to secure it to the user's body. A mount (not shown) attachable to the user and mateable with data processing unit 102 may be used. For example, a mount may include an adhesive surface. Data processing unit 102 performs data processing functions, where such functions may include but are not limited to, filtering and encoding of data signals, each of which corresponds to a sampled analyte level of the user, for transmission to primary receiver unit 104 via the communication link 103. In one embodiment, sensor 101 or data processing unit 102 or a combined sensor/data processing unit may be wholly implantable under the skin layer of the user.

In certain embodiments, primary receiver unit 104 may include an analog interface section including an RF receiver and an antenna that is configured to communicate with data processing unit 102 via the communication link 103, and a data processing section for processing the received data from data processing unit 102 such as data decoding, error detection and correction, data clock generation, data bit recovery, etc., or any combination thereof.

In operation, primary receiver unit 104 in certain embodiments is configured to synchronize with data processing unit 102 to uniquely identify data processing unit 102, based on, for example, an identification information of data processing unit 102, and thereafter, to periodically receive signals transmitted from data processing unit 102 associated with the monitored analyte levels detected by sensor 101.

Referring again to FIG. 1, data processing terminal 105 may include a personal computer, a portable computer such as a laptop or a handheld device (e.g., personal digital assistants (PDAs), telephone such as a cellular phone (e.g., a multimedia and Internet-enabled mobile phone such as an iPhone or similar phone), mp3 player, pager, and the like), drug delivery device, each of which may be configured for data communication with the receiver via a wired or a wireless connection. Additionally, data processing terminal 105 may further be connected to a data network (not shown) for storing, retrieving, updating, and/or analyzing data corresponding to the detected analyte level of the user.

In certain embodiments, data processing terminal 105 may include an infusion device such as an insulin infusion pump or the like, which may be configured to administer insulin to patients, and which may be configured to communicate with primary receiver unit 104 for receiving, among others, the measured analyte level. Alternatively, primary receiver unit 104 may be configured to integrate an infusion device therein so that primary receiver unit 104 is configured to administer insulin (or other appropriate drug) therapy to patients, for example, for administering and modifying basal profiles, as well as for determining appropriate boluses for administration based on, among others, the detected analyte levels received from data processing unit 102. An infusion device may be an external device or an internal device (wholly implantable in a user).

In certain embodiments, data processing terminal 105, which may include an insulin pump, may be configured to receive the analyte signals from data processing unit 102, and thus, incorporate the functions of primary receiver unit 104 including data processing for managing the patient's insulin therapy and analyte monitoring. In certain embodiments, the communication link 103 as well as one or more of the other communication interfaces shown in FIG. 1, may use one or more of: an RF communication protocol, an infrared communication protocol, a Bluetooth® enabled communication protocol, an 802.11x wireless communication protocol, or an equivalent wireless communication protocol which would allow secure, wireless communication of several units (for example, per HIPAA requirements), while avoiding potential data collision and interference.

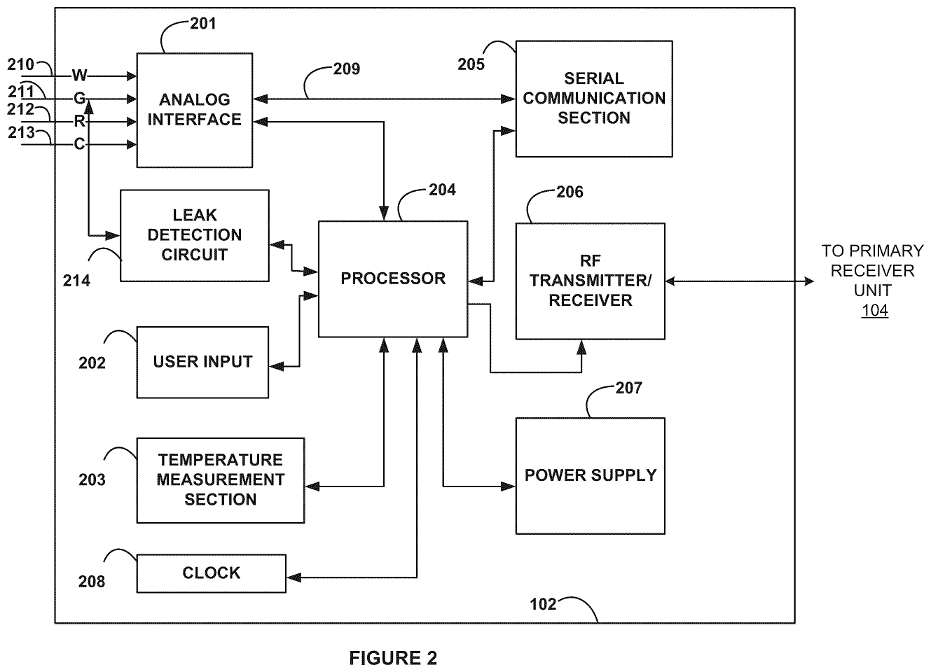

FIG. 2 shows a block diagram of an embodiment of a data processing unit of the data monitoring and detection system shown in FIG. 1. User input and/or interface components may be included or a data processing unit may be free of user input and/or interface components. In certain embodiments, one or more ASICs may be used to implement one or more functions or routines associated with the operations of the data processing unit (and/or receiver unit) using for example one or more state machines and buffers. Referring to the Figure, the data processing unit 102 in one embodiment includes an analog interface 201 configured to communicate with the sensor 101 (FIG. 1), a user input 202, and a temperature measurement section 203, each of which is operatively coupled to a processor 204 such as a central processing unit (CPU).

As can be seen in the embodiment of FIG. 2, the sensor 101 (FIG. 1) includes four contacts, three of which are electrodes-work electrode (W) 210, reference electrode (R) 212, and counter electrode (C) 213, each operatively coupled to the analog interface 201 of data processing unit 102. This embodiment also shows optional guard contact (G) 211. Fewer or greater electrodes may be employed. For example, the counter and reference electrode functions may be served by a single counter/reference electrode, there may be more than one working electrode and/or reference electrode and/or counter electrode, etc.

The electronics of the on-skin sensor control unit and the sensor are operated using a power supply 207, e.g., a battery. Additionally, as can be seen from the Figure, clock 208 is provided to, among others, supply real time information to the processor 204.

In one aspect, the analog interface 201 is coupled via the conductive contacts of data processing unit 102 to one or more sensors 101. The analog interface 201 in one embodiment is configured to receive signals from and to operate the sensor(s). For example, in one embodiment, the analyte interface 201 may obtain signals from sensor 101 (FIG. 1) using amperometric, coulometric, potentiometric, voltammetric, and/or other electrochemical techniques. For example, to obtain amperometric measurements, the analog interface 201 includes a potentiostat that provides a constant potential to sensor 101. In other embodiments, the analog interface 201 includes an amperostat that supplies a constant current to a sensor and can be used to obtain coulometric or potentiometric measurements.

The signal from the sensor 101 (FIG. 1) generally has at least one characteristic, such as, for example, current, voltage, or frequency, or the like, which varies with the concentration of the analyte that the sensor 101 is monitoring. For example, if the analog interface 201 operates using amperometry, then the current signal from the sensor varies with variation in the monitored analyte concentration. Referring back to FIG. 2, one or more of the components of data processing unit 102, including, for example, the processor 204, the analog interface 201, and/or the RF transmitter/receiver 206 may include circuitry which converts the information-carrying portion of the signal from one characteristic to another. For example, the one or more components of data processing unit 102 such as the processor 204, the analyte interface 201, and/or the RF transmitter/receiver 206 may include a current-to-voltage or current-to-frequency converter. In one aspect, the converter may be configured to provide a signal that is, for example, more easily transmitted, readable by digital circuits, and/or less susceptible to noise contributions. In one embodiment, a unidirectional input path is established from the sensor 101 (FIG. 1) and/or manufacturing and testing equipment to the analog interface 201 of the data processing unit 102, while a unidirectional output is established from the output of the RF transmitter 206 of the data processing unit 102 for transmission to the primary receiver unit 104. In this manner, a data path is shown in FIG. 2 between the aforementioned unidirectional input and output via a dedicated link 209 from the analog interface 201 to serial communication section 205, thereafter to the processor 204, and then to the RF transmitter 206. Also shown is a leak detection circuit 214 coupled to the guard contact (G) 211 and the processor 204 in the data processing unit 102. The leak detection circuit 214 in accordance with one embodiment of the present invention may be configured to detect leakage current in the sensor 101 to determine whether the measured sensor data are corrupt or whether the measured data from the sensor 101 is accurate.

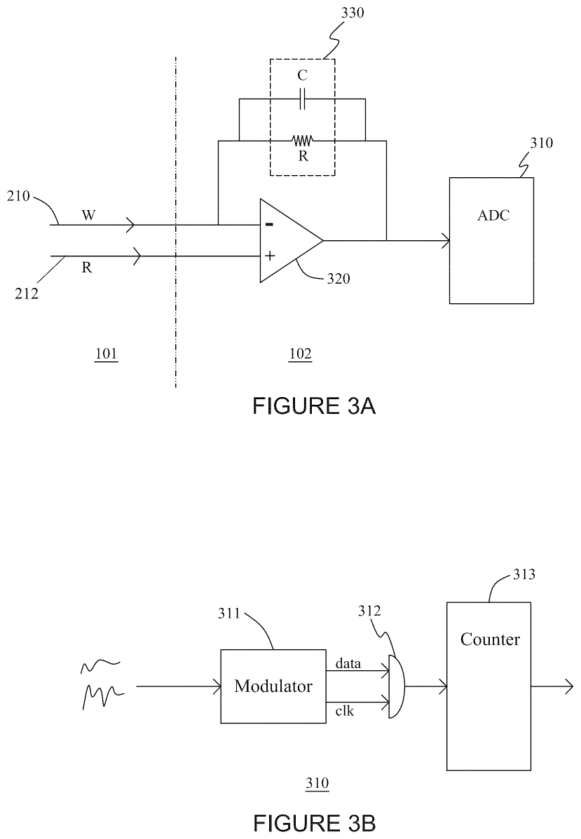

Referring now to FIGS. 3A and 3B, the sensor interface electronics including the analog interface section of the data processing unit 102 (FIG. 1). More specifically, referring to FIG. 3A, there is provided an analog to digital converter (ADC) 310 which is configured to receive the filtered output analog signal of the operational amplifier 320 which has one of its input terminals (negative input terminal as shown, for example) operatively coupled to the working electrode W 210 of sensor 101 (FIG. 1). As described, in one aspect, the operational amplifier 320 is configured to maintain the voltage level at the working electrode W 210 at approximately two volts while the reference electrode R 212 (FIG. 2) of the sensor (FIG. 1) is operatively coupled to the positive input terminal of the operational amplifier 320 and maintained at approximately 1.96 Volts, so that a difference of approximately 40 m Volts is maintained as the poise voltage to power the sensor electrodes.

Referring back to FIG. 3A, the filter 330 operatively coupled between the negative input terminal and the output terminal of the operational amplifier 320, is configured to filter the received signal from the sensor electrodes to remove or filter out the signal transients. Signal artifacts may be limited to a predetermined signal bandwidth (for example, to approximately 0.1 Hz) effectively operating as a bandpass filter prior to providing the signal to the analog to digital converter ADC 310 for further processing.

In one embodiment, the resistor R of the filter 330 has a resistance value of approximately 1.58 MOhms, while the capacitor C has a capacitance of approximately one microFarad. While specific values are provided herein, it is to be noted that these values for the various components of the filter as well as the operational amplifier are for exemplary purposes only, and are not intended to limit the scope of the embodiments of the present disclosure to such particular values or parameters. Within the scope of the present disclosure, other values or parameters are contemplated for example, for the resistor R and the capacitor C of the filter 330, as well as the operational amplifier 320.

Referring still to FIG. 3A, the filtered output signal from the operational amplifier 320 in the form of an analog voltage signal is provided to the analog to digital converter 310 for further processing as described herein. That is, referring now to FIG. 3B, a detailed description of a portion of the ADC 310 is shown. More specifically, as shown in FIG. 3B, the ADC 310 includes, among others, a signal modulator 311 operatively coupled to the input of the ADC 310 to receive the voltage signal from the output of the operational amplifier 320 (FIG. 3A). The signal modulator in one embodiment may include a Sigma-Delta modulator. A Sigma-Delta modulator, in certain embodiments, allows for high resolution analog-to-digital conversion achievable utilizing low cost circuits. In other embodiments, different or modified modulators with similar or equivalent functionality may be provided including, for example, converters, filters, and/or signal processing devices and components.

In one aspect, the modulator 311 is configured to modulate the received analog voltage signal from the output of the operational amplifier 320 to generate a corresponding frequency data stream which is synchronized with one or more clock signals. That is, referring to FIG. 3B, in one aspect, the modulator 311 is configured to convert the received voltage signal which may vary between, for example, 1.5 Volts to 2.0 Volts, into a corresponding frequency data stream. In one aspect, the modulator 311 includes a servo loop and the output of the modulator 311 includes the converted or modulated frequency data stream and the corresponding clock signal.

Referring again to FIG. 3B, AND gate 312 is shown and provided to the ADC 310, and is configured to receive the frequency data stream (shown as “data” in FIG. 3B) and the clock signal (shown as “clk” in FIG. 3B) and perform an AND function to synchronize the received frequency data with the clock signal to generate a corresponding frequency pulse to provide to a counter 313. That is, as shown in FIG. 3B, the output terminal of the AND gate 312 is operatively coupled to the counter 313, and the counter 313, in one embodiment, is configured to count the pulses received from the AND gate 312. In some embodiments, the analog to digital conversion is conducted quickly, in order to obtain a converted signal for a specific point in time. In order for the analog to digital conversion to be achieved substantially immediately, a high frequency clock signal is used, such that the counted pulses of the conversion occur in rapid succession.

In other embodiments, a lower frequency clock, for example a 1024 Hz clock signal, may be implemented and the analog to digital conversion is spread out over a predetermined or programmed time period, such as, for example, a 30 second (or other suitable or appropriate) time period or other suitable measurement time period. In one aspect, the counter 313 is configured to receive the synchronized frequency data stream or pulses from the AND gate 312 and counts the received pulses over the predetermined time period to generate an average accumulated count, that is representative of the average data associated with the signal from sensor 101 (FIG. 1) over the time period.

In one aspect, the clock signal used to synchronize the data stream from the modulator 311 is a 1024 Hz signal. Furthermore, in one embodiment, the AND gate 312 operates such that the pulse from the modulator 311 is received by the counter 313 when the clock signal is a “1” (as opposed to a “0”). In this manner, in one aspect, the counter 313 is initially set to a zero count, and thereafter, when the predetermined measurement time period has elapsed (for example, the 30 second time period), the counter 313 has accumulated pulses during this time duration to output an average value of the accumulated pulses received from the time duration as the corresponding sensor data processed from the voltage signal received from the working electrode 210 (FIG. 2) of sensor 101 (FIG. 1).

In this manner, in one aspect, lower power consumption of the data processing unit 102 (FIG. 1) may be achieved, in addition to improving processed analyte signal resolution as well as providing a built-in filtering function to filter out signal transients and/or noise associated with the received signals. That is, given that the counter is configured to accumulate the pulses over an extended measurement time period such as 30 seconds or other suitable time period, and given that the determined or programmed time period defines the filter frequency response, by customizing or tailoring the time period to a particular sensor or sensors, the frequency response associated with the sensor may be improved. Moreover, the predetermined or programmed time period provides a time duration which allows the use of low frequency modulator clock (e.g., 1024 Hz) which consumes less power, and thus, extends the power supply life of the data processing unit 102 (FIG. 1).

Referring still again to FIGS. 3A and 3B, in the manner described above, in aspects of the present disclosure, an extended measurement time period is provided based on, for example, the predetermined or programmed time period to sample or accumulate the pulses or data stream received from analyte sensor 101, and that is determined by or is a function of the clock discussed above in conjunction with the modulator and configured to establish the maximum resolution as a function of the conversion (analog to digital) time. In this manner, in one aspect, an extended sampling time period (as compared to a discrete signal sampling) is provided to extend the signal conversion process over a longer measurement time period resulting in improved power consumption management, higher signal resolution and noise or artifact filtered analyte signals for further processing, including transmission to a remote device or location for presentation or output to the user or patient.

Accumulating or averaging the received frequency data stream or pulses from analyte sensor 101 results in a frequency domain filter function that rolls off at a frequency equal to the reciprocal of the signal conversion time. Thus, any frequency or components of the data stream that is a multiple of the signal conversion time is rejected, filtering out undesirable signal artifacts, among others. Additionally, given that common signal frequency interference occurs at the power line frequencies of 50 and 60 Hz, and multiples thereof, by modifying or adjusting the conversion time duration to be a division of the common interference frequencies, the filter function is configured to remove these frequency signals.

Additionally, as discussed above, the embodiments described above provides low power consumption by providing a slow modulator clock while retaining the desired high signal resolution, as the slow clock consumes a low amount of power such that, using 1024 Hz clock with a 30 second conversion time period, a 15 bit signal resolution may be attained. In aspects of the present disclosure, the clock function of the modulator 311 may be a divided down clock as described above, divided down from a 32.768 KHz crystal to the 1024 Hz clock.

Referring back to FIGS. 3A and 3B, the converted analyte related signal from the output of the ADC 310 is further processed for storage, additional filtering, and data packing, encoding and the like for wireless transmission, for example to the receiver/monitor unit 104, as well as other data processing routines described herein.

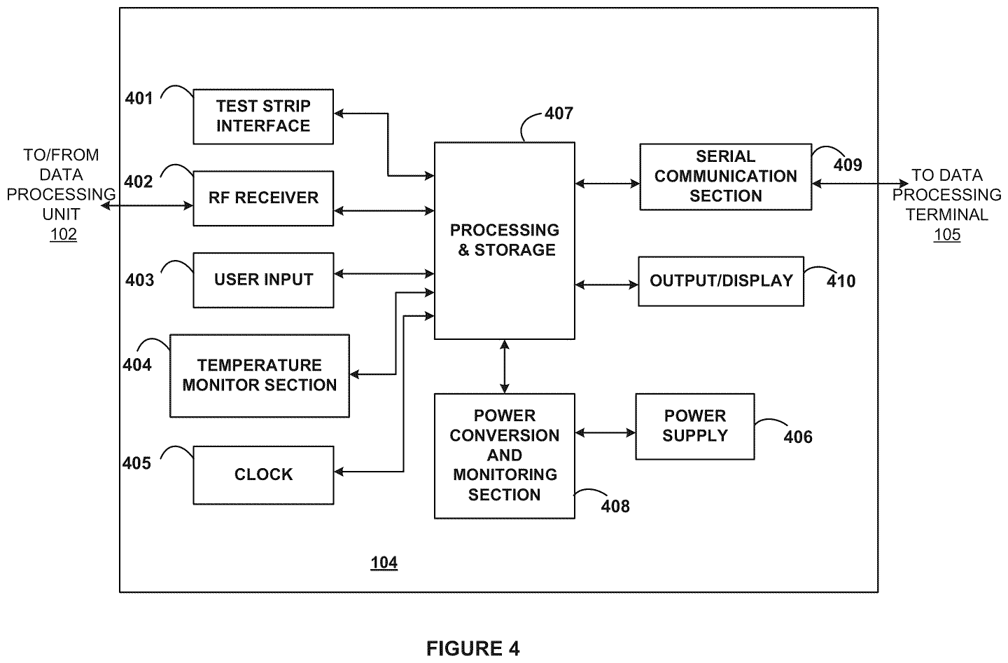

FIG. 4 is a block diagram of an embodiment of a receiver/monitor unit such as primary receiver unit 104 of the data monitoring and management system shown in FIG. 1. In certain embodiments, primary receiver unit 104 includes one or more of: a blood glucose test strip interface 401, an RF receiver 402, a user input 403, a temperature monitor section 404, and a clock 405, each of which is operatively coupled to a processing and storage section 407. Primary receiver unit 104 also includes a power supply 406 operatively coupled to a power conversion and monitoring section 408. Further, the power conversion and monitoring section 408 is also coupled to the receiver processing and storage section 407. Moreover, also shown are a receiver serial communication section 409, and an output 410, each operatively coupled to the processing and storage unit 407. The receiver may include user input and/or interface components or may be free of user input and/or interface components.

In certain embodiments, the test strip interface 401 includes a glucose level testing portion to receive a blood (or other body fluid sample) glucose test or information related thereto. For example, the interface may include a test strip port to receive a glucose test strip. The device may determine the glucose level of the test strip, and optionally display (or otherwise notice) the glucose level on the output 410 of primary receiver unit 104. Any suitable test strip may be employed, e.g., test strips that only require a very small amount (e.g., one microliter or less, e.g., 0.5 microliter or less, e.g., 0.1 microliter or less), of applied sample to the strip in order to obtain accurate glucose information, e.g. FreeStyle® blood glucose test strips from Abbott Diabetes Care Inc. Glucose information obtained by the in vitro glucose testing device may be used for a variety of purposes, computations, etc. For example, the information may be used to calibrate sensor 101, confirm results of sensor 101 to increase the confidence thereof (e.g., in instances in which information obtained by sensor 101 is employed in therapy related decisions), etc.

In further embodiments, data processing unit 102 and/or primary receiver unit 104 and/or the secondary receiver unit 106, and/or the data processing terminal/infusion section 105 may be configured to receive the blood glucose value wirelessly over a communication link from, for example, a blood glucose meter. In further embodiments, a user manipulating or using the analyte monitoring system 100 (FIG. 1) may manually input the blood glucose value using, for example, a user interface (for example, a keyboard, keypad, voice commands, and the like) incorporated in the one or more of data processing unit 102, primary receiver unit 104, secondary receiver unit 106, or the data processing terminal/infusion section 105.

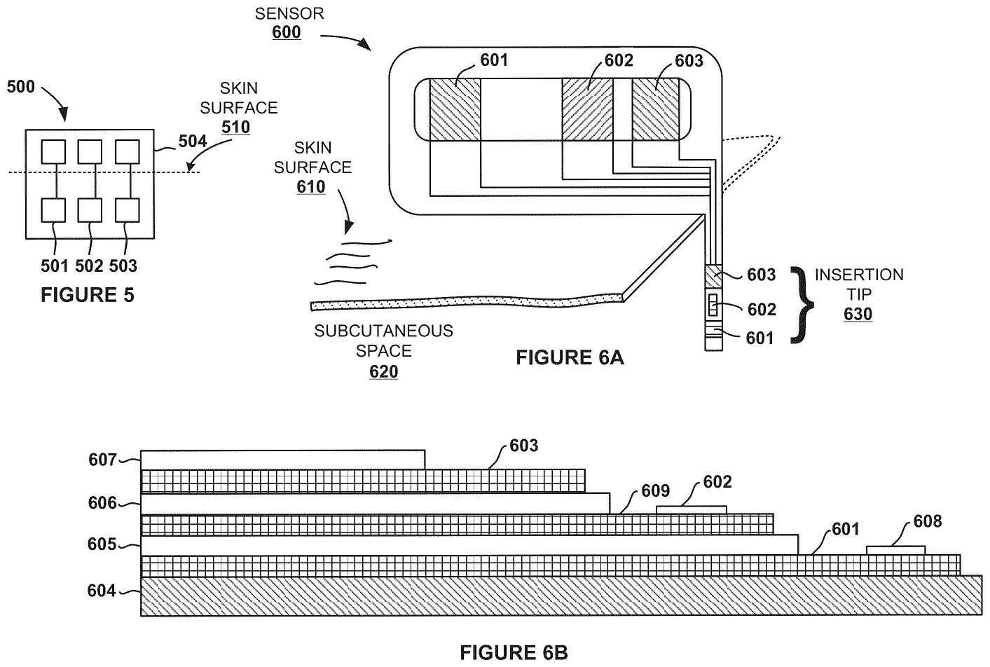

FIG. 5 schematically shows an embodiment of an analyte sensor in accordance with the present disclosure. This sensor embodiment includes electrodes 501, 502 and 503 on a base 504. Electrodes (and/or other features) may be applied or otherwise processed using any suitable technology, e.g., chemical vapor deposition (CVD), physical vapor deposition, sputtering, reactive sputtering, photolithography, printing, coating, ablating (e.g., laser ablation), painting, dip coating, etching, and the like. Materials include but are not limited to aluminum, carbon (such as graphite), cobalt, copper, gallium, gold, indium, iridium, iron, lead, magnesium, mercury (as an amalgam), nickel, niobium, osmium, palladium, platinum, rhenium, rhodium, selenium, silicon (e.g., doped polycrystalline silicon), silver, tantalum, tin, titanium, tungsten, uranium, vanadium, zinc, zirconium, mixtures thereof, and alloys, oxides, or metallic compounds of these elements.

The sensor may be wholly implantable in a user or may be configured so that only a portion is positioned within (internal) a user and another portion outside (external) a user. For example, the sensor 500 may include a portion positionable above a surface of the skin 510, and a portion positioned below the skin. In such embodiments, the external portion may include contacts (connected to respective electrodes of the second portion by traces) to connect to another device also external to the user such as a transmitter unit. While the embodiment of FIG. 5 shows three electrodes side-by-side on the same surface of base 504, other configurations are contemplated, e.g., fewer or greater electrodes, some or all electrodes on different surfaces of the base or present on another base, some or all electrodes stacked together, electrodes of differing materials and dimensions, etc.

FIG. 6A shows a perspective view of an embodiment of an electrochemical analyte sensor 600 having a first portion (which in this embodiment may be characterized as a major portion) positionable above a surface of the skin 610, and a second portion (which in this embodiment may be characterized as a minor portion) that includes an insertion tip 630 positionable below the skin, e.g., penetrating through the skin and into, e.g., the subcutaneous space 620, in contact with the user's biofluid such as interstitial fluid. Contact portions of a working electrode 601, a reference electrode 602, and a counter electrode 603 are positioned on the portion of the sensor 600 situated above the skin surface 610. Working electrode 601, a reference electrode 602, and a counter electrode 603 are shown at the second section and particularly at the insertion tip 630. Traces may be provided from the electrode at the tip to the contact, as shown in FIG. 6A. It is to be understood that greater or fewer electrodes may be provided on a sensor. For example, a sensor may include more than one working electrode and/or the counter and reference electrodes may be a single counter/reference electrode, etc.

FIG. 6B shows a cross sectional view of a portion of the sensor 600 of FIG. 6A. The electrodes 601, 602 and 603, of the sensor 600 as well as the substrate and the dielectric layers are provided in a layered configuration or construction. For example, as shown in FIG. 6B, in one aspect, the sensor 600 (such as the sensor 101 FIG. 1), includes a substrate layer 604, and a first conducting layer 601 such as carbon, gold, etc., disposed on at least a portion of the substrate layer 604, and which may provide the working electrode. Also shown disposed on at least a portion of the first conducting layer 601 is a sensing layer 608.

A first insulation layer such as a first dielectric layer 605 is disposed or layered on at least a portion of the first conducting layer 601, and further, a second conducting layer 609 may be disposed or stacked on top of at least a portion of the first insulation layer (or dielectric layer) 605. As shown in FIG. 6B, the second conducting layer 609 may provide the reference electrode 602, and in one aspect, may include a layer of silver/silver chloride (Ag/AgCl), gold, etc.

A second insulation layer 606 such as a dielectric layer in one embodiment may be disposed or layered on at least a portion of the second conducting layer 609. Further, a third conducting layer 603 may provide the counter electrode 603. It may be disposed on at least a portion of the second insulation layer 606. Finally, a third insulation layer 607 may be disposed or layered on at least a portion of the third conducting layer 603. In this manner, the sensor 600 may be layered such that at least a portion of each of the conducting layers is separated by a respective insulation layer (for example, a dielectric layer). The embodiment of FIGS. 6A and 6B show the layers having different lengths. Some or all of the layers may have the same or different lengths and/or widths.

In certain embodiments, some or all of the electrodes 601, 602, 603 may be provided on the same side of the substrate 604 in the layered construction as described above, or alternatively, may be provided in a co-planar manner such that two or more electrodes may be positioned on the same plane (e.g., side-by side (e.g., parallel) or angled relative to each other) on the substrate 604. For example, co-planar electrodes may include a suitable spacing there between and/or include dielectric material or insulation material disposed between the conducting layers/electrodes. Furthermore, in certain embodiments one or more of the electrodes 601, 602, 603 may be disposed on opposing sides of the substrate 604. In such embodiments, contact pads may be on the same or different sides of the substrate. For example, an electrode may be on a first side and its respective contact may be on a second side, e.g., a trace connecting the electrode and the contact may traverse through the substrate.

As noted above, analyte sensors may include an analyte-responsive enzyme to provide a sensing component or sensing layer. Some analytes, such as oxygen, can be directly electrooxidized or electroreduced on a sensor, and more specifically at least on a working electrode of a sensor. Other analytes, such as glucose and lactate, require the presence of at least one electron transfer agent and/or at least one catalyst to facilitate the electrooxidation or electroreduction of the analyte. Catalysts may also be used for those analytes, such as oxygen, that can be directly electrooxidized or electroreduced on the working electrode. For these analytes, each working electrode includes a sensing layer (see for example sensing layer 608 of FIG. 6B) proximate to or on a surface of a working electrode. In many embodiments, a sensing layer is formed near or on only a small portion of at least a working electrode.

The sensing layer includes one or more components designed to facilitate the electrochemical oxidation or reduction of the analyte. The sensing layer may include, for example, a catalyst to catalyze a reaction of the analyte and produce a response at the working electrode, an electron transfer agent to transfer electrons between the analyte and the working electrode (or other component), or both.

A variety of different sensing layer configurations may be used. In certain embodiments, the sensing layer is deposited on the conductive material of a working electrode. The sensing layer may extend beyond the conductive material of the working electrode. In some cases, the sensing layer may also extend over other electrodes, e.g., over the counter electrode and/or reference electrode (or counter/reference is provided).

A sensing layer that is not in direct contact with the working electrode may include a catalyst that facilitates a reaction of the analyte. However, such sensing layers may include an electron transfer agent that transfers electrons directly from the analyte to the working electrode, as the sensing layer is spaced apart from the working electrode. One example of this type of sensor is a glucose or lactate sensor which includes an enzyme (e.g., glucose oxidase, glucose dehydrogenase, lactate oxidase, and the like) in the sensing layer. The glucose or lactate may react with a second compound in the presence of the enzyme. The second compound may then be electrooxidized or electroreduced at the electrode. Changes in the signal at the electrode indicate changes in the level of the second compound in the fluid and are proportional to changes in glucose or lactate level and, thus, correlate to the analyte level.

In certain embodiments which include more than one working electrode, one or more of the working electrodes may not have a corresponding sensing layer, or may have a sensing layer which does not contain one or more components (e.g., an electron transfer agent and/or catalyst) needed to electrolyze the analyte. Thus, the signal at this working electrode may correspond to background signal which may be removed from the analyte signal obtained from one or more other working electrodes that are associated with fully-functional sensing layers by, for example, subtracting the signal.

In certain embodiments, the sensing layer includes one or more electron transfer agents. Electron transfer agents that may be employed are electroreducible and electrooxidizable ions or molecules having redox potentials that are a few hundred millivolts above or below the redox potential of the standard calomel electrode (SCE). The electron transfer agent may be organic, organometallic, or inorganic. Examples of organic redox species are quinones and species that in their oxidized state have quinoid structures, such as Nile blue and indophenol. Examples of organometallic redox species are metallocenes such as ferrocene. Examples of inorganic redox species are hexacyanoferrate (III), ruthenium hexamine etc.

In certain embodiments, electron transfer agents have structures or charges which prevent or substantially reduce the diffusional loss of the electron transfer agent during the period of time that the sample is being analyzed. For example, electron transfer agents include but are not limited to a redox species, e.g., bound to a polymer which can in turn be disposed on or near the working electrode. The bond between the redox species and the polymer may be covalent, coordinative, or ionic. Although any organic, organometallic or inorganic redox species may be bound to a polymer and used as an electron transfer agent, in certain embodiments the redox species is a transition metal compound or complex, e.g., osmium, ruthenium, iron, and cobalt compounds or complexes. It will be recognized that many redox species described for use with a polymeric component may also be used, without a polymeric component.

One type of polymeric electron transfer agent contains a redox species covalently bound in a polymeric composition. An example of this type of mediator is poly(vinylferrocene). Another type of electron transfer agent contains an ionically-bound redox species. This type of mediator may include a charged polymer coupled to an oppositely charged redox species. Examples of this type of mediator include a negatively charged polymer coupled to a positively charged redox species such as an osmium or ruthenium polypyridyl cation. Another example of an ionically-bound mediator is a positively charged polymer such as quaternized poly(4-vinyl pyridine) or poly(1-vinyl imidazole) coupled to a negatively charged redox species such as ferricyanide or ferrocyanide. In other embodiments, electron transfer agents include a redox species coordinatively bound to a polymer. For example, the mediator may be formed by coordination of an osmium or cobalt 2,2′-bipyridyl complex to poly(1-vinyl imidazole) or poly(4-vinyl pyridine).

Suitable electron transfer agents are osmium transition metal complexes with one or more ligands, each ligand having a nitrogen-containing heterocycle such as 2,2′-bipyridine, 1,10-phenanthroline, 1-methyl, 2-pyridyl biimidazole, or derivatives thereof. The electron transfer agents may also have one or more ligands covalently bound in a polymer, each ligand having at least one nitrogen-containing heterocycle, such as pyridine, imidazole, or derivatives thereof. One example of an electron transfer agent includes (a) a polymer or copolymer having pyridine or imidazole functional groups and (b) osmium cations complexed with two ligands, each ligand containing 2,2′-bipyridine, 1,10-phenanthroline, or derivatives thereof, the two ligands not necessarily being the same. Some derivatives of 2,2′-bipyridine for complexation with the osmium cation include but are not limited to 4,4′-dimethyl-2,2′-bipyridine and mono-, di-, and polyalkoxy-2,2′-bipyridines, such as 4,4′-dimethoxy-2,2′-bipyridine. Derivatives of 1,10-phenanthroline for complexation with the osmium cation include but are not limited to 4,7-dimethyl-1,10-phenanthroline and mono, di-, and polyalkoxy-1,10-phenanthrolines, such as 4,7-dimethoxy-1,10-phenanthroline. Polymers for complexation with the osmium cation include but are not limited to polymers and copolymers of poly(1-vinyl imidazole) (referred to as “PVI”) and poly(4-vinyl pyridine) (referred to as “PVP”). Suitable copolymer substituents of poly(1-vinyl imidazole) include acrylonitrile, acrylamide, and substituted or quaternized N-vinyl imidazole, e.g., electron transfer agents with osmium complexed to a polymer or copolymer of poly(1-vinyl imidazole).

Embodiments may employ electron transfer agents having a redox potential ranging from about −200 mV to about +200 mV versus the standard calomel electrode (SCE). The sensing layer may also include a catalyst which is capable of catalyzing a reaction of the analyte. The catalyst may also, in some embodiments, act as an electron transfer agent. One example of a suitable catalyst is an enzyme which catalyzes a reaction of the analyte. For example, a catalyst, such as a glucose oxidase, glucose dehydrogenase (e.g., pyrroloquinoline quinone (PQQ), dependent glucose dehydrogenase, flavine adenine dinucleotide (FAD), or nicotinamide adenine dinucleotide (NAD) dependent glucose dehydrogenase), may be used when the analyte of interest is glucose. A lactate oxidase or lactate dehydrogenase may be used when the analyte of interest is lactate. Laccase may be used when the analyte of interest is oxygen or when oxygen is generated or consumed in response to a reaction of the analyte.

The sensing layer may also include a catalyst which is capable of catalyzing a reaction of the analyte. The catalyst may also, in some embodiments, act as an electron transfer agent. One example of a suitable catalyst is an enzyme which catalyzes a reaction of the analyte. For example, a catalyst, such as a glucose oxidase, glucose dehydrogenase (e.g., pyrroloquinoline quinone (PQQ), dependent glucose dehydrogenase or oligosaccharide dehydrogenase, flavine adenine dinucleotide (FAD) dependent glucose dehydrogenase, nicotinamide adenine dinucleotide (NAD) dependent glucose dehydrogenase), may be used when the analyte of interest is glucose. A lactate oxidase or lactate dehydrogenase may be used when the analyte of interest is lactate. Laccase may be used when the analyte of interest is oxygen or when oxygen is generated or consumed in response to a reaction of the analyte.

In certain embodiments, a catalyst may be attached to a polymer, cross linking the catalyst with another electron transfer agent (which, as described above, may be polymeric). A second catalyst may also be used in certain embodiments. This second catalyst may be used to catalyze a reaction of a product compound resulting from the catalyzed reaction of the analyte. The second catalyst may operate with an electron transfer agent to electrolyze the product compound to generate a signal at the working electrode. Alternatively, a second catalyst may be provided in an interferent-eliminating layer to catalyze reactions that remove interferents.

Certain embodiments include a Wired Enzyme™ sensing layer (Abbott Diabetes Care Inc.) that works at a gentle oxidizing potential, e.g., a potential of about +40 mV. This sensing layer uses an osmium (Os)-based mediator designed for low potential operation and is stably anchored in a polymeric layer. Accordingly, in certain embodiments the sensing element is a redox active component that includes (1) Osmium-based mediator molecules attached by stable (bidente) ligands anchored to a polymeric backbone, and (2) glucose oxidase enzyme molecules. These two constituents are crosslinked together.

A mass transport limiting layer (not shown), e.g., an analyte flux modulating layer, may be included with the sensor to act as a diffusion-limiting barrier to reduce the rate of mass transport of the analyte, for example, glucose or lactate, into the region around the working electrodes. The mass transport limiting layers are useful in limiting the flux of an analyte to a working electrode in an electrochemical sensor so that the sensor is linearly responsive over a large range of analyte concentrations and is easily calibrated. Mass transport limiting layers may include polymers and may be biocompatible. A mass transport limiting layer may provide many functions, e.g., biocompatibility and/or interferent-eliminating, etc.

In certain embodiments, a mass transport limiting layer is a membrane composed of crosslinked polymers containing heterocyclic nitrogen groups, such as polymers of polyvinylpyridine and polyvinylimidazole. Embodiments also include membranes that are made of a polyurethane, or polyether urethane, or chemically related material, or membranes that are made of silicone, and the like.

A membrane may be formed by crosslinking in situ a polymer, modified with a zwitterionic moiety, a non-pyridine copolymer component, and optionally another moiety that is either hydrophilic or hydrophobic, and/or has other desirable properties, in an alcohol-buffer solution. The modified polymer may be made from a precursor polymer containing heterocyclic nitrogen groups. For example, a precursor polymer may be polyvinylpyridine or polyvinylimidazole. Optionally, hydrophilic or hydrophobic modifiers may be used to “fine-tune” the permeability of the resulting membrane to an analyte of interest. Optional hydrophilic modifiers, such as poly(ethylene glycol), hydroxyl or polyhydroxyl modifiers, may be used to enhance the biocompatibility of the polymer or the resulting membrane.

A membrane may be formed in situ by applying an alcohol-buffer solution of a crosslinker and a modified polymer over an enzyme-containing sensing layer and allowing the solution to cure for about one to two days or other appropriate time period. The crosslinker-polymer solution may be applied to the sensing layer by placing a droplet or droplets of the solution on the sensor, by dipping the sensor into the solution, or the like. Generally, the thickness of the membrane is controlled by the concentration of the solution, by the number of droplets of the solution applied, by the number of times the sensor is dipped in the solution, or by any combination of these factors. A membrane applied in this manner may have any combination of the following functions: (1) mass transport limitation, i.e., reduction of the flux of analyte that can reach the sensing layer, (2) biocompatibility enhancement, or (3) interferent reduction.

The electrochemical sensors may employ any suitable measurement technique. For example, may detect current or may employ potentiometry. Technique may include, but are not limited to, amperometry, coulometry, and voltammetry. In some embodiments, sensing systems may be optical, colorimetric, and the like.

In certain embodiments, the sensing system detects hydrogen peroxide to infer glucose levels. For example, a hydrogen peroxide-detecting sensor may be constructed in which a sensing layer includes enzyme such as glucose oxides, glucose dehydrogensae, or the like, and is positioned proximate to the working electrode. The sensing layer may be covered by a membrane that is selectively permeable to glucose. Once the glucose passes through the membrane, it is oxidized by the enzyme and reduced glucose oxidase can then be oxidized by reacting with molecular oxygen to produce hydrogen peroxide.

Certain embodiments include a hydrogen peroxide-detecting sensor constructed from a sensing layer prepared by crosslinking two components together, for example: (1) a redox compound such as a redox polymer containing pendent Os polypyridyl complexes with oxidation potentials of about +200 mV vs. SCE, and (2) periodate oxidized horseradish peroxidase (HRP). Such a sensor functions in a reductive mode; the working electrode is controlled at a potential negative to that of the Os complex, resulting in mediated reduction of hydrogen peroxide through the HRP catalyst.

In another example, a potentiometric sensor can be constructed as follows. A glucose-sensing layer is constructed by crosslinking together (1) a redox polymer containing pendent Os polypyridyl complexes with oxidation potentials from about −200 mV to +200 mV vs. SCE, and (2) glucose oxidase. This sensor can then be used in a potentiometric mode, by exposing the sensor to a glucose containing solution, under conditions of zero current flow, and allowing the ratio of reduced/oxidized Os to reach an equilibrium value. The reduced/oxidized Os ratio varies in a reproducible way with the glucose concentration, and will cause the electrode's potential to vary in a similar way.

A sensor may also include an active agent such as an anticlotting and/or antiglycolytic agent(s) disposed on at least a portion of a sensor that is positioned in a user. An anticlotting agent may reduce or eliminate the clotting of blood or other body fluid around the sensor, particularly after insertion of the sensor. Examples of useful anticlotting agents include heparin and tissue plasminogen activator (TPA), as well as other known anticlotting agents. Embodiments may include an antiglycolytic agent or precursor thereof. Examples of antiglycolytic agents are glyceraldehyde, fluoride ion, and mannose.

Sensors may be configured to require no system calibration or no user calibration. For example, a sensor may be factory calibrated and need not require further calibrating. In certain embodiments, calibration may be required, but may be done without user intervention, i.e., may be automatic. In those embodiments in which calibration by the user is required, the calibration may be according to a predetermined schedule or may be dynamic, i.e., the time for which may be determined by the system on a real-time basis according to various factors, such as but not limited to glucose concentration and/or temperature and/or rate of change of glucose, etc.

Calibration may be accomplished using an in vitro test strip (or other reference), e.g., a small sample test strip such as a test strip that requires less than about 1 microliter of sample (for example FreeStyle® blood glucose monitoring test strips from Abbott Diabetes Care Inc.). For example, test strips that require less than about 1 nanoliter of sample may be used. In certain embodiments, a sensor may be calibrated using only one sample of body fluid per calibration event. For example, a user need only lance a body part one time to obtain sample for a calibration event (e.g., for a test strip), or may lance more than one time within a short period of time if an insufficient volume of sample is firstly obtained. Embodiments include obtaining and using multiple samples of body fluid for a given calibration event, where glucose values of each sample are substantially similar. Data obtained from a given calibration event may be used independently to calibrate or combined with data obtained from previous calibration events, e.g., averaged including weighted averaged, etc., to calibrate. In certain embodiments, a system need only be calibrated once by a user, where recalibration of the system is not required.

Analyte systems may include an optional alarm system that, e.g., based on information from a processor, warns the patient of a potentially detrimental condition of the analyte. For example, if glucose is the analyte, an alarm system may warn a user of conditions such as hypoglycemia and/or hyperglycemia and/or impending hypoglycemia, and/or impending hyperglycemia. An alarm system may be triggered when analyte levels approach, reach or exceed a threshold value. An alarm system may also, or alternatively, be activated when the rate of change, or acceleration of the rate of change, in analyte level increase or decrease approaches, reaches or exceeds a threshold rate or acceleration. A system may also include system alarms that notify a user of system information such as battery condition, calibration, sensor dislodgment, sensor malfunction, etc. Alarms may be, for example, auditory and/or visual. Other sensory-stimulating alarm systems may be used including alarm systems which heat, cool, vibrate, or produce a mild electrical shock when activated.

The subject disclosure also includes sensors used in sensor-based drug delivery systems. The system may provide a drug to counteract the high or low level of the analyte in response to the signals from one or more sensors. Alternatively, the system may monitor the drug concentration to ensure that the drug remains within a desired therapeutic range. The drug delivery system may include one or more (e.g., two or more) sensors, a processing unit such as a transmitter, a receiver/display unit, and a drug administration system. In some cases, some or all components may be integrated in a single unit. A sensor-based drug delivery system may use data from the one or more sensors to provide necessary input for a control algorithm/mechanism to adjust the administration of drugs, e.g., automatically or semi-automatically. As an example, a glucose sensor may be used to control and adjust the administration of insulin from an external or implanted insulin pump.

In certain embodiments, an analyte signal processing method may include determining a measurement time period, receiving a plurality of signals associated with a monitored analyte level during the determined measurement time period from an analyte sensor, modulating the received plurality of signals to generate a data stream over the measurement time period, and accumulating the generated data stream to determine an analyte signal corresponding to the monitored analyte level associated with the measurement time period.

Certain aspects may further include filtering the received plurality of signals associated with the monitored analyte level.

In certain embodiments, filtering may include bandpass filtering the plurality of signals.

In certain embodiments, modulating the received plurality of signals may include synchronizing each of the received plurality of signals with a corresponding clock signal to generate a respective frequency pulse.

In certain embodiments, the accumulated data stream may include maintaining a count of the number of generated frequency pulses during the measurement time period.

In certain embodiments, the determined analyte signal corresponding to the monitored analyte level may include an average value of the generated frequency pulses.

In certain embodiments, the received plurality of signals may be voltage signals, and further, modulating the generated data stream may include converting each of the voltage signals into a corresponding frequency pulse stream.

In certain aspects, the average of the frequency pulse stream for the measurement time period may correspond to the determined analyte level for the measurement time period.

In certain embodiments, the measurement time period may be programmable.

In certain embodiments, the measurement time period may be modified based, at least in part, on one or more characteristics of the analyte sensor.

Certain aspects may further include removing one or more signal artifacts from the received plurality of signals.

In certain embodiments, removing the one or more signal artifacts may include filtering the received plurality of signals prior to modulating the signals.

In certain embodiments, a signal processing device used for processing analyte related signals may include an analyte sensor interface electronics for receiving a plurality of analyte related signals from an analyte sensor over a measurement time period, and a data processing component operatively coupled to the analyte sensor interface electronics for processing the received plurality of analyte related signals, the data processing component including, a signal filtering component to filter the received plurality of analyte related signals, and a signal conversion component operatively coupled to the signal filtering component to convert the received filtered plurality of analyte related signals to determine an analyte level associated with a monitored analyte level during the measurement time period.

In certain embodiments, at least a portion of the one or more analyte sensor interface electronics or the data processing components may include an application specific integrated circuit.

In certain embodiments, the signal conversion component may include a clock to generate a plurality of clock signals.

In certain embodiments, the signal conversion component may synchronize each of the received filtered plurality of analyte related signals to the corresponding one of the generated plurality of clock signals.

In certain embodiments, the signal conversion component may generate a frequency data stream based on the received filtered plurality of analyte related signals.

In certain embodiments, each pulse in the frequency data stream may be associated with the respective one of the generated plurality of clock signals.

In certain embodiments, the signal conversion component may include a sigma delta modulation unit.

Certain aspects may include a communication component to transmit data associated with the determined analyte level over a communication connection.

In certain embodiments, the communication component may include an RF transmitter.

In certain embodiments, the communication connection may include one or more of a wired communication link or a wireless communication link.

In certain embodiments, the analyte sensor may include a glucose sensor.

In certain embodiments, the glucose sensor may include at least one working electrode in signal communication with the analyte sensor interface electronics.

In certain embodiments, the working electrode of the sensor may include one of carbon or gold.

In certain embodiments, an analyte monitoring system may include an analyte sensor including a working electrode having a sensing layer at least a portion of which is configured for fluid contact with an interstitial fluid under a skin layer, and a data processing unit comprising an application specific integrated circuit in signal communication with the analyte sensor for receiving a plurality of signals related to a monitored analyte level from the sensor over a monitoring time period, the data processing unit including a signal filtering component to filter the received plurality of signals, and a signal conversion component to convert the received filtered plurality of analyte related signals to determine a corresponding analyte level associated with a monitored analyte level during a measurement time period, said monitoring time period including multiple measurement time periods.

In certain embodiments, the signal conversion component may include a clock to generate a plurality of clock signals.

In certain embodiments, the signal conversion component may synchronize each of the received filtered plurality of analyte related signals to the corresponding one of the generated plurality of clock signals.

In certain embodiments, the signal conversion component may generate a frequency data stream based on the received filtered plurality of analyte related signals.

In certain embodiments, each pulse in the frequency data stream may be associated with the respective one of the generated plurality of clock signals.

In certain embodiments, the data processing unit may include a sigma delta modulator.

In certain embodiments, the analyte sensor may include a glucose sensor.

In certain embodiments, the working electrode of the sensor may include one of carbon or gold.

In certain embodiments, the sensing layer may include redox polymer.

In certain embodiments, the working electrode may be provided on the analyte sensor using laser ablation or photolithography.

Certain aspects may further include a receiver unit in signal communication with the data processing unit to receive a plurality of data corresponding to the monitored analyte level from the sensor or the data processing unit.

In certain embodiments, the receiver unit may include a strip port to receive an in vitro glucose test strip.

In certain embodiments, the analyte sensor may be calibrated based on the glucose measurement derived from the in vitro test strip.

In certain embodiments, the measurement time period may be approximately 30 seconds.

In certain embodiments, the monitoring time period may be between approximately five to seven days.

Various other modifications and alterations in the structure and method of operation of the embodiments of the present disclosure will be apparent to those skilled in the art without departing from the scope and spirit of the present disclosure. Although the present disclosure has been described in connection with certain embodiments, it should be understood that the present disclosure as claimed should not be unduly limited to such embodiments. It is intended that the following claims define the scope of the present disclosure and that structures and methods within the scope of these claims and their equivalents be covered thereby.

1. An in vivo glucose monitoring system comprising a sensor control device, the sensor control device comprising:
a glucose sensor, wherein at least a portion of the glucose sensor is configured to be in fluid contact with a bodily fluid of a subject;
wireless communication circuitry adapted to wirelessly transmit data;
one or more processors; and
memory on which one or more instructions are stored that, when executed by the one or more processors, cause the one or more processors to:
determine a measurement time period;
generate a plurality of clock signals using a clock;
receive a plurality of analog signals associated with a monitored glucose level during the determined measurement time period from the glucose sensor, the measurement time period establishing a maximum signal resolution associated with the clock and a sigma-delta modulator as a function of analog to digital conversion time to obtain a higher signal resolution;
modulating the received plurality of analog signals using the sigma-delta modulator to generate a frequency data stream over the measurement time period based on the received plurality of analog signals by synchronizing each of the received plurality of analog signals to a corresponding one of the generated plurality of clock signals, and filtering one or more signal artifacts from the frequency data stream over the measurement time period, the one or more signal artifacts including a signal transient; and
accumulating the generated frequency data stream to determine a glucose signal corresponding to the monitored glucose level associated with the measurement time period.

2. The in vivo glucose monitoring system of claim 1, wherein the frequency data stream includes one or more pulses each associated with a respective one of the generated plurality of clock signals.

3. The in vivo glucose monitoring system of claim 1, wherein the one or more instructions, when executed by the one or more processors, further cause the one or more processors to:
transmit data associated with the monitored glucose level over a communication connection.

4. The in vivo glucose monitoring system of claim 1, wherein the received plurality of analog signals are current signals.

5. The in vivo glucose monitoring system of claim 1, wherein the one or more instructions, when executed by the one or more processors, further cause the one or more processors to:
average the accumulated generated frequency data stream over the measurement time period.

6. The in vivo glucose monitoring system of claim 1, wherein the glucose sensor comprises a plurality of electrodes including a working electrode comprising an analyte-responsive enzyme bonded to a polymer disposed on the working electrode.

7. The in vivo glucose monitoring system of claim 1, wherein the glucose sensor comprises a plurality of electrodes including a working electrode comprising a mediator bonded to a polymer disposed on the working electrode.

8. The in vivo glucose monitoring system of claim 1, wherein the glucose sensor includes at least one working electrode in signal communication with an analyte sensor interface electronics.

9. The in vivo glucose monitoring system of claim 3, wherein the communication connection includes an RF communication connection.

10. The in vivo glucose monitoring system of claim 3, wherein the communication connection includes one or more of a wired communication link or a wireless communication link.

11. The in vivo glucose monitoring system of claim 6, wherein the analyte-responsive enzyme is chemically bonded to the polymer.

12. The in vivo glucose monitoring system of claim 6, wherein the working electrode further comprises a mediator.

13. The in vivo glucose monitoring system of claim 7, wherein the mediator is chemically bonded to the polymer.

14. The in vivo glucose monitoring system of claim 7, wherein the mediator is crosslinked with the polymer.

15. The in vivo glucose monitoring system of claim 1, wherein the glucose sensor includes at least one working electrode and the at least one working electrode comprises one of carbon or gold.
